(19) United States
(12) Patent Application Publication
(10) Pub. No.: US 2023/0057759 A1
(43) Pub. Date: Feb. 23, 2023

(54) Solid State Form of Pyroxasulfone

(71) Applicant: ADAMA AGAN LTD., Ashdod (IL)

(72) Inventors: Judith Aronhime, Rehovot (IL); Rotem Sella-Erez, Tel Aviv (IL)

(73) Assignee: ADAMA AGAN LTD., Ashdod (IL)

(21) Appl. No.: 17/792,306

(22) PCT Filed: Jan. 13, 2021
(86) PCT No.: PCT/IL2021/050042

§ 371 Date: Jul. 12, 2022

Related U.S. Application Data
(60) Provisional application No. 62/961,233, filed on Jan. 15, 2020.

(51) Int. Cl. A01N 43/80; C07D 413/12; A01P 13/02

(52) U.S. Cl. CPC A01N 43/80; C07D 413/12; A01P 13/02; C07B 2200/13

(57) ABSTRACT

The present disclosure relates to a solid state form of Pyroxasulfone, processes for preparation thereof and agrochemical compositions thereof.

### RELATED APPLICATION

This application claims the benefit of priority of U.S. Provisional Patent Application No. 62/961,233 filed on 15 Jan. 2020, the contents of which are incorporated herein by reference in their entirety.

### TECHNICAL FIELD

The present disclosure relates to solid state form of Pyroxasulfone, processes for preparation thereof and agrochemical compositions thereof.

### BACKGROUND OF THE DISCLOSURE

Pyroxasulfone has the chemical name 3-[[5-(difluoromethoxy)-1-methyl-3-(trifluoromethyl)pyrazol-4-yl[methylsulfonyl]-5,5-dimethyl-4H-1,2-oxazole.

Pyroxasulfone has the following chemical structure:

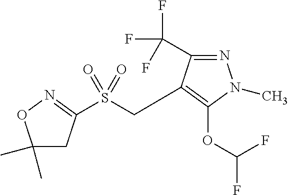

Pyroxasulfone is a novel pre-emergence herbicide for wheat, corn, and soybean. Pyroxasulfone inhibits the biosynthesis of very-long-chain fatty acids in plants and has shown excellent herbicidal activity against grass and broadleaf weeds at lower application rates compared with other commercial herbicides. Pyroxasulfone is a pre-emergence herbicide to control grass and small-seeded broadleaf weeds. In fields of genetically modified crops, Pyroxasulfone controlled weeds that were resistant to non-selective herbicides. Pyroxasulfone has been classified in the Herbicide Resistance Action Committee Group K3, and inhibits the biosynthesis of very-long-chain fatty acids in plants.

Pyroxasulfone is first disclosed by Kumiai Chemical Industry Co., Ltd. and Ihara Chemical Industry Co., Ltd. in the U.S. Pat. No. 7,238,689. No solid state form of Pyroxasulfone was disclosed in the literature.

Polymorphism, the occurrence of different crystal forms, is a property of some molecules and molecular complexes. A single compound, like Pyroxasulfone, may give rise to a variety of polymorphs having distinct crystal structures and physical properties like melting point, thermal behaviours (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C—) NMR spectrum. One or more of these techniques may be used to distinguish different polymorphic forms of a compound.

Different salts and solid state forms (including solvated forms) of an active ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final formulation or recipe, for instance, if they serve to improve dissolution. Different salts and solid state forms and solvates of an active ingredient may also give rise to a variety of polymorphs or crystalline forms, which may in turn provide additional opportunities to use variations in the properties and characteristics of a solid active ingredient for providing an improved product.

Discovering new salts, solid state forms and solvates of an agrochemical product can provide materials having desirable processing properties, such as ease of handling, ease of processing, storage stability, and ease of purification or as desirable intermediate crystal forms that facilitate conversion to other salts or polymorphic forms. New salts, polymorphic forms and solvates of an active ingredient can also provide an opportunity to improve the performance characteristics of an agrochemical product (dissolution profile, permeability, etc.). It expands the range of materials that an expert in the field can avail for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for some solid state forms (including solvated forms or salts) of Pyroxasulfone.

### SUMMARY

The present disclosure relates to a solid state form of Pyroxasulfone, processes for preparation thereof, and agrochemical compositions including this solid state form.

The present disclosure also provides use of the solid state form of Pyroxasulfone for preparing other solid state forms of Pyroxasulfone, Pyroxasulfone salts and solid state forms of a Pyroxasulfone salt.

The present disclosure also provides solid state form of Pyroxasulfone of the present disclosure for uses in the preparation of other solid state forms of Pyroxasulfone, Pyroxasulfone salts and solid state forms of a Pyroxasulfone salt.

In one embodiment, the present disclosure encompasses the described solid state forms of Pyroxasulfone for use in the preparation of agrochemical compositions and/or formulations, optionally for cross spectrum activity against grasses and broadleaf weeds, to be applied at the pre-emergence timings.

In another embodiment, the present disclosure encompasses uses of the described solid state forms of Pyroxasulfone for the preparation of agrochemical compositions and/or formulations.

The present disclosure further provides agrochemical compositions including one or more solid state forms of Pyroxasulfone according to the present disclosure.

In yet another embodiment, the present disclosure encompasses agrochemical formulations including one or more of the described solid state forms of Pyroxasulfone and at least one agrochemically acceptable excipient and safener.

The present disclosure encompasses processes to prepare said agrochemical formulations of Pyroxasulfone including one or more of the described solid state forms and at least one agrochemically acceptable excipient and safener.

The solid state forms defined herein as well as the agrochemical compositions or formulations of the solid state forms of Pyroxasulfone can be used as an herbicide for the selective control of grasses and broadleaf weeds in the crops.

The present disclosure also provides methods for weed control comprising applying to one or both the weeds and their habitat an effective amount of a solid state form of Pyroxasulfone.

The present disclosure also provides uses of the solid state form of Pyroxasulfone of the present disclosure, or at least one of the above agrochemical compositions or formulations for the selective control of grasses and broadleaf weeds in the crops.

### BRIEF DESCRIPTION OF THE SEVERAL VIEWS OF THE DRAWING(S)

FIG. 1 shows an X-ray powder diffractogram (XRPD) of Form A of Pyroxasulfone.

### DETAILED DESCRIPTION

The present disclosure relates to solid state forms of Pyroxasulfone, processes for preparation thereof and agrochemical compositions including this solid state form. The disclosure also relates to the conversion of the described solid state form of Pyroxasulfone to other solid state forms of Pyroxasulfone, Pyroxasulfone salts and their solid state forms thereof.

The solid state forms of Pyroxasulfone according to the present disclosure may have advantageous properties selected from at least one of: chemical or polymorphic purity, flowability, solubility, dissolution rate, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data as depicted in a FIGURE. Such data include, for example, powder X-ray diffractograms and solid state NMR spectra. As is well-known in the art, the graphical data potentially provides additional technical information to further define the respective solid state form which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the FIGURES herein with graphical data generated for an unknown crystal form and confirm whether the two sets of graphical data are characterizing the same crystal form or two different crystal forms. A crystal form of Pyroxasulfone referred to herein as being characterized by graphical data “as depicted in” a FIGURE will thus be understood to include any crystal form of the Pyroxasulfone, characterized with the graphical data having such small variations, as are well known to the skilled person, in comparison with the FIGURE.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or 0% of any other forms of the subject compound as measured, for example, by XRPD. Thus, the solid state form of Pyroxasulfone described herein as substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Pyroxasulfone. Accordingly, in some embodiments of the disclosure, the described solid state form of Pyroxasulfone may contain from about 1% to about 20% (w/w), from about 5% to about 20% (w/w), or from about 5% to about 10% (w/w) of one or more other solid state forms of the same Pyroxasulfone.

As used herein, unless stated otherwise, XRPD peaks reported herein are optionally measured using CuK_{α}, radiation, λ=1.54187 Å.

As used herein, the term “isolated” in reference to solid state forms of Pyroxasulfone of the present disclosure corresponds to solid state form of Pyroxasulfone that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

The present disclosure includes a solid state form of Pyroxasulfone designated as Form A.

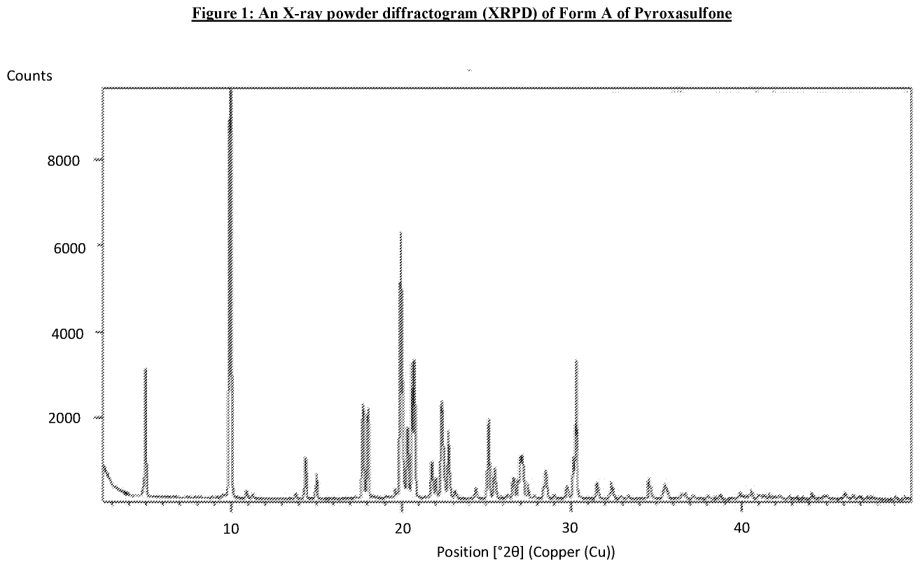

The Form A of Pyroxasulfone can be characterized by data selected from one or more of the following: an XRPD pattern having peaks at 5.0, 10.0, 20.0, 25.1 and 30.3 degrees 2-theta±0.2 degrees 2-theta; an XRPD pattern as depicted in FIG. 1; and combinations of these data.

Crystalline Form A of Pyroxasulfone may be further characterized by the XRPD pattern having peaks at 5.0, 10.0, 20.0, 25.1 and 30.3 degrees 2-theta±0.2 degrees 2-theta, and also having one, two or three additional peaks selected from 17.8, 20.9 and 22.4 degrees 2-theta±0.2 degrees 2-theta.

Crystalline Form A of Pyroxasulfone may be characterized by an XRPD pattern as depicted in FIG. 1.

Crystalline Form A of Pyroxasulfone may be characterized by any combination of at least four peaks in an XRPD pattern as depicted in FIG. 1.

The present disclosure also provides the solid state forms of Pyroxasulfone of the present disclosure for use in the preparation of other solid state forms of Pyroxasulfone, Pyroxasulfone salts and solid state forms thereof.

The present disclosure further encompasses processes for preparing Pyroxasulfone solid state form. The process includes starting from Pyroxasulfone, and converting it to a Pyroxasulfone solid state form. The conversion can be done, for example, by a process including subjecting a solution of Pyroxasulfone in a solvent system followed by decantation and drying, wherein the solvent system is selected from a series of solvents comprising Methanol, ethyl acetate, Tetrahydrofuran (THF), 2-Methyl THF, dichloromethane, n-Heptane, Propyl acetate, Toluene, and combinations thereof.

In another embodiment, the present disclosure encompasses the above described solid state forms of Pyroxasulfone for use in the preparation of agrochemical compositions and/or formulations, optionally for cross spectrum activity against grasses and broadleaf weeds, to be applied at the pre-emergence timings.

In another embodiment, the present disclosure encompasses the use of the described solid state form of Pyroxasulfone for the preparation of agrochemical compositions and/or formulations.

The present disclosure further provides agrochemical compositions including one or more solid state forms of Pyroxasulfone according to the present disclosure.

In yet another embodiment, the present disclosure encompasses agrochemical formulations including one or more of the described solid state forms of Pyroxasulfone and at least one agrochemically acceptable safener.

In an embodiment, the present disclosure encompasses processes to prepare said agrochemical formulations of Pyroxasulfone including one or more of the described solid state forms and at least one agrochemically acceptable safener.

The solid state forms defined herein as well as the agrochemical compositions or formulations of the solid state forms of Pyroxasulfone can be used as an herbicide for the selective control of grasses and broadleaf weeds in the crops.

The present disclosure also provides methods for weed control comprising applying to one or both the weeds and their habitat an effective amount of a solid state form of Pyroxasulfone.

The present disclosure also provides uses of the solid state forms of Pyroxasulfone of the present disclosure, or at least one of the above agrochemical compositions or formulations for the selective control of grasses and broadleaf weeds in the crops.

Having described the disclosure with reference to certain preferred embodiments, other embodiments will become apparent to one skilled in the art from consideration of the specification. The disclosure is further illustrated by reference to the following examples describing in detail the preparation of the solid forms and methods of use of the disclosure. It will be apparent to those skilled in the art that many modifications, both to materials and methods, may be practiced without departing from the scope of the disclosure.

### Analytical Methods

### X-Ray Powder Diffraction Method:

Powder X-ray Diffraction was performed on an X-Ray powder diffractometer PanAlytical X′pert Pro; CuK_{α}, radiation, (λ=1.54187 Å-); laboratory temperature 25±3° C.; zero background sample holders.

Measurement parameters:
Scan range: 3-40 degrees 2-theta
Scan mode: continuous
Step size: 0.013 degrees
Sample holder: zero background silicon plate
Prior to analysis, the samples were gently ground using a mortar and pestle to obtain a fine powder. Optionally, silicon powder can be added in a suitable amount as internal standard in order to calibrate the positions of the diffractions. The ground sample was adjusted into a cavity of the sample holder and the surface of the sample was smoothed using a cover.

### EXAMPLES

Pyroxasulfone was prepared according to the following general procedure:

### Example 1: General Procedure for Pyroxasulfone

A reactor was charged with 94 Kg of MeOH, 17 Kg of 3-[[5-(difluoromethoxy)-1-methyl-3-(trifluoromethyl)pyrazol-4-yl]methylsulfanyl]-5,5-dimethyl-4H-isoxazole and 482 g of Sodium Tungstate at 20° C., and the reaction mixture was heated to 50° C. An aqueous solution of 28% Hydrogen peroxide (11.2 Kg) was added dropwise in 1 h and the reaction mixture was stirred at 50° C. for 4 h. An aqueous solution of 28% Hydrogen peroxide (1.6 Kg) was added dropwise (15 min) and the reaction mixture was stirred for additional 3 h at 50° C., while monitoring the reaction progress. pH was corrected to pH=8.8-9 with 5% NaOH at 50° C. An aqueous solution of 50% sodium thiosulfate was added to decompose excess of H₂O₂ (Checked with QUANTOFIX peroxide 100 test strips) and water was added slowly (18 ml). Reaction mixture was heated to 70° C. and stirred for 30 min (pH corrected to 8.8 if needed). Reaction mixture was cooled down to 10° C. during 1 h to precipitate the reaction product, which was filtered and washed with MeOH (13.5 Kg), followed by with water (13.5 Kg). The cake was slurried with water (170 Kg), filtered and washed again with water (80 Kg). The solid product, Pyroxasulfone, was dried under vacuum at 50° C. Solid was then ground with spatula and analysed by XRD.

Form A of Pyroxasulfone was also prepared according to following preparatory examples:

### Example 2: Preparation of Pyroxasulfone Form A

About 100 mg of Pyroxasulfone was taken in a 4 ml glass vial and ethyl acetate (1 ml) was added. The vial was shaken on an Eppendorf ThermoMixer C at 800 RPM/25° C. for 5 minutes and then a clear solution was observed. The solution was slowly cooled to 0° C. and left standing for 3 hours at 0° C. The obtained solid was removed from the solvent by decantation and dried at ambient condition. Solid was then ground with spatula and analysed by XRD.

### Example 3: Preparation of Pyroxasulfone Form A

About 100 mg of Pyroxasulfone was taken in a 4 ml glass vial and THF (0.7 ml) was added. The mixture was stirred at room temperature until clear solution was observed. The solution was slowly cooled to 0° C. and left standing for 3 hours at 0° C. The obtained solid was removed from the solvent by decantation and dried at ambient condition. Solid was then ground with spatula and analysed by XRD.

### Example 4: Preparation of Pyroxasulfone Form A

About 100 mg of Pyroxasulfone was taken in a 4 ml glass vial and dichloromethane (0.7 ml) was added. The mixture was stirred at room temperature until clear solution was observed. Then n-Heptane was added slowly to the solution until precipitation was obtained. The precipitated solid was removed from the solvent by centrifugation and dried at ambient condition. Solid was then ground with spatula and analysed by XRD.

### Example 5: Preparation of Pyroxasulfone Form A

About 100 mg of Pyroxasulfone was taken in a 10 ml round bottle flask and propyl acetate (1 ml) was added. The mixture was stirred and heated to 102° C. for 12 hours. A clear solution was observed, and the flask was slowly cooled to room temperature and then to 2-8° C. for 2 hours. The mixture was then kept in an ice tray at 0° C. for additional 2 hours. The obtained solid was removed from the solvent by decantation and dried at ambient condition. Solid was then ground with spatula and analysed by XRD.

### Example 6: Preparation of Pyroxasulfone Form A

About 100 mg of Pyroxasulfone was taken in a 10 ml round bottle flask and toluene (1 ml) was added. The mixture was stirred and heated to 110° C. for 12 hours. A clear solution was observed and the flask was slowly cooled to room temperature and then to 2-8° C. for 2 hours. The mixture was then kept in an ice tray at 0° C. for additional 2 hours. The obtained solid was removed from the solvent by decantation and dried at ambient condition. Solid was then ground with spatula and analysed by XRD.

### Example 7: Preparation of Pyroxasulfone Form A

About 100 mg of Pyroxasulfone was taken in a 4 ml glass vial and 2 ml of 2-Methyl THF was added. The vial was shaken on an Eppendorf ThermoMixerC at 800 RPM/25° C. for 5 minutes until a clear solution was obtained. The vial was left open for slow evaporation under ambient condition for 2 days at room temperature. The obtained crystals were collected and air dried under hood. The solid was ground gently in a mortar with a pestle and then analysed by XRD.

In addition, any priority document(s) of this application is/are hereby incorporated herein by reference in its/their entirety.

What is claimed is:

1. A Crystalline Form A of Pyroxasulfone, which is characterized by data selected from one or more of the following:
i. an XRPD pattern having peaks at 5.0, 10.0, 20.0, 25.1 and 30.3 degrees 2-theta±0.2 degrees 2-theta;
ii. an XRPD pattern as depicted in FIG. 1.

2. The crystalline form of Pyroxasulfone according to claim 1, which is characterized by an XRPD pattern having peaks at 5.0, 10.0, 20.0, 25.1 and 30.3 degrees 2-theta±0.2 degrees 2-theta, and also having one, two or three additional peaks selected from 17.8, 20.9 and 22.4 degrees 2-theta±0.2 degrees 2-theta.

3. An agrochemical composition comprising a crystalline form according to any one of claims 1-2.

4. Use of a crystalline form according to any one of claims 1-2 in the preparation of an agrochemical composition and/or formulation.

5. An agrochemical formulation comprising a crystalline form according to any one of claims 1-2 or an agrochemical composition of claim 3, and at least one agrochemically acceptable excipient.

6. A crystalline form according to any one of claims 1 to 2, an agrochemical composition according to claim 3, or an agrochemical formulation according to claim 5, for the use as an herbicide for the selective control of grasses and broadleaf weeds in crops.

7. A crystalline form according to any one of claims 1 to 2, an agrochemical composition according to claim 3, or an agrochemical formulation according to claim 5, for the use in the cross spectrum activity against grasses and broadleaf weeds, applied at the pre-emergence timings.

8. Use of a crystalline form according to any one of claims 1 to 2, an agrochemical composition according to claim 3, or an agrochemical formulation according to claim 5, for the use as an herbicide for the selective control of grasses and broadleaf weeds in crops, preferably for the use in the cross spectrum activity against grasses and broadleaf weeds, applied at the pre-emergence timings.

9. A process of preparation of the crystalline form of Pyroxasulfone according to claim 1 or claim 2, comprising subjecting a solution of Pyroxasulfone in a solvent system followed by decantation and drying.

10. The process of preparation of the crystalline form of Pyroxasulfone according to claim 9, comprising subjecting a solution of Pyroxasulfone in a solvent system, wherein the solvent system is selected from a series of solvents comprising Methanol, ethyl acetate, Tetrahydrofuran (THF), 2-Methyl THF, dichloromethane, n-Heptane, Propyl acetate, Toluene, and combinations thereof.
